(19) United States
(12) Patent Application Publication
(10) Pub. No.: US 2019/0247105 A1
(43) Pub. Date: Aug. 15, 2019

(54) RESIDUAL FLUID MEASUREMENT SYSTEM AND METHOD

(71) Applicant: Cryterion Medical, Inc., Carlsbad, CA (US)

(72) Inventor: Chadi Harmouche, Saint-Laurent (CA)

(21) Appl. No.: 16/275,567

(22) Filed: Feb. 14, 2019

Related U.S. Application Data
(60) Provisional application No. 62/630,707, filed on Feb. 14, 2018.

(51) Int. Cl. A61B 18/02; G01F 9/00; G01F 1/34

(52) U.S. Cl. CPC A61B 18/02; A61B 2018/00577; G01F 1/34; G01F 9/00

(57) ABSTRACT

A cryoablation system comprises a balloon catheter including a cryoballoon, and a fluid container configured to supply cryogenic fluid to the cryoballoon and to store a residual fluid quantity. A first fluid sensor senses the residual fluid quantity and generates a first sensor output. A second fluid sensor senses fluid consumption information representing an amount of fluid consumption by the balloon catheter during one or more ablation procedures, and generates a second sensor output. A controller receives the first sensor output and the second sensor output and determines residual cryoablation information based at least in part on the first sensor output and the second sensor output.

### CROSS-REFERENCE TO RELATED APPLICATION

This application claims priority to Provisional Application No. 62/630,707, filed Feb. 14, 2018, which is herein incorporated by reference in its entirety.

### TECHNICAL FIELD

The present disclosure relates to medical devices and methods for treating cardiac arrhythmias. More specifically, the disclosure relates to devices and methods for cardiac cryoablation.

### BACKGROUND

Cardiac arrhythmias involve an abnormality in the electrical conduction of the heart and are a leading cause of stroke, heart disease, and sudden cardiac death. Treatment options for patients with arrhythmias include medications and/or the use of medical devices, which can include implantable devices and/or catheter ablation of cardiac tissue, to name a few. In particular, catheter ablation involves delivering ablative energy to tissue inside the heart to block aberrant electrical activity from depolarizing heart muscle cells out of synchrony with the heart's normal conduction pattern. The procedure is performed by positioning the tip of an energy delivery catheter adjacent to diseased or target tissue in the heart. The energy delivery component of the system is typically at or near the most distal (i.e. farthest from the operator or user) portion of the catheter, and often at the tip of the catheter.

Various forms of energy can be used to ablate diseased heart tissue. One form of energy that is used to ablate diseased heart tissue includes cryogenics (also referred to herein as “cryoablation”). During a cryoablation procedure, the tip of the catheter is positioned adjacent to target cardiac tissue, at which time energy is delivered in the form of a refrigerant or cryogenic fluid to create tissue necrosis, rendering the ablated tissue incapable of conducting electrical signals.

Cryosurgical, and in particular, catheter-based cryoablation systems consume various cryogenic fluids (e.g., liquid nitrous oxide or liquid nitrogen) that are typically provided in high-pressure fluid containers in either liquid or gas form (collectively referred to herein as “cryogenic fluid”). During cryoablation procedures, it is often difficult to accurately assess whether there is a sufficient quantity of cryogenic fluid remaining within the fluid container to successfully complete other cryoablation procedures. However, it is appreciated that these fluid containers should have sufficient cryogenic fluid therein in order to successfully complete such procedures. Complete depletion of the cryogenic fluid during the cryoablation procedure would not only interrupt the procedure, but it could also be injurious to the patient.

### SUMMARY

In Example 1, a cryoablation system comprises a balloon catheter including a cryoballoon, and a fluid container configured to supply cryogenic fluid to the cryoballoon and to store a residual fluid quantity. A first fluid sensor senses the residual fluid quantity and generates a first sensor output. A second fluid sensor senses fluid consumption information representing an amount of fluid consumption by the balloon catheter during one or more ablation procedures, and generates a second sensor output. A controller receives the first sensor output and the second sensor output and determines residual cryoablation information based at least in part on the first sensor output and the second sensor output.

In Example 2, the cryoablation system of Example 1, wherein the residual cryoablation information includes a number of cryoablation procedures that may be performed with the residual fluid quantity.

In Example 3, the cryoablation system of Example 1, wherein the residual cryoablation information includes an amount of time available to perform one or more cryoablation procedures using the residual fluid quantity.

In Example 4, the cryoablation system of any of Examples 1-3, wherein the first fluid sensor includes a pressure sensor or a weight sensor.

In Example 5, the cryoablation system of any of Examples 1-4, wherein the second fluid sensor includes a flow rate sensor.

In Example 6, the cryoablation system of any of Examples 1-5, wherein the fluid consumption information includes an actual fluid flow rate.

In Example 7, the cryoablation system of any of Examples 1-5, wherein the fluid consumption information includes an average fluid flow rate.

In Example 8, the cryoablation system of any of Examples 1-7, further comprising a graphical user interface including a display configured to display the residual cryoablation information.

In Example 9, a method for determining residual cryoablation information for a fluid container having a residual fluid quantity. The method comprises the steps of generating a first sensor output with a first fluid sensor, sending the first sensor output to a controller, and determining the residual cryoablation information with the controller based at least in part upon the first sensor output.

In Example 10, the method of Example 9, wherein the residual cryoablation information includes a number of cryoablation procedures that may be performed with the residual fluid quantity.

In Example 11, the method of Example 9, wherein the residual cryoablation information includes an amount of time to perform cryoablation procedures with the residual fluid quantity.

In Example 12, the method of any of Examples 9-11, further comprising generating a second sensor output with a second fluid sensor.

In Example 13, the method of Example 12, further comprising the step of sending the second sensor output generated by the second connection sensor to the controller.

In Example 14, the method of Example 13, further comprising the step of determining the residual cryoablation information based at least partially upon the second sensor output with the controller.

In Example 15, the method of any of Examples 9-14, further comprising displaying the residual cryoablation information on a display of a graphical user interface.

In Example 16, a residual fluid measurement system for determining residual cryoablation information for a fluid container having a residual fluid quantity. The residual fluid measurement system comprises a first fluid sensor that senses the residual fluid quantity and generates a first sensor output, and a controller that receives the first sensor output and determines the residual cryoablation information based at least in part on the first sensor output.

In Example 17, the residual fluid measurement system of Example 16, wherein the residual cryoablation information includes a number of cryoablation procedures that may be performed.

In Example 18, the residual fluid measurement system of Example 16, wherein the residual cryoablation information includes an amount of time available to perform one or more cryoablation procedures.

In Example 19, the residual fluid measurement system of Example 16, further comprising a second fluid sensor that senses fluid consumption information, the second fluid sensor generating a second sensor output.

In Example 20, the residual fluid measurement system of Example 19, wherein the controller receives the second sensor output, the controller determining the residual cryoablation information based at least in part on the second sensor output.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic view of a patient and one embodiment of a cryogenic balloon catheter system including an embodiment of a residual fluid measurement system having features of the present disclosure;

FIG. 2 is a simplified schematic side view of a portion of an embodiment of the cryogenic balloon catheter system including another embodiment of the residual fluid measurement system;

FIG. 3 is a simplified schematic side view of a portion of another embodiment of the cryogenic balloon catheter system including still another embodiment of the residual fluid measurement system; and

FIG. 4 is a simplified schematic side view of a portion of still another embodiment of the cryogenic balloon catheter system including yet another embodiment of the residual fluid measurement system.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

Embodiments of the present disclosure are described herein in the context of a residual fluid measurement system. Those of ordinary skill in the art will realize that the following detailed description of the present disclosure is illustrative only and is not intended to be in any way limiting. Other embodiments of the residual fluid measurement system will readily suggest themselves to such skilled persons having the benefit of this disclosure. Reference will now be made in detail to implementations of the present disclosure as illustrated in the accompanying drawings.

In the interest of clarity, not all of the routine features of the implementations described herein are shown and described. It will, of course, be appreciated that in the development of any such actual implementation, numerous implementation-specific decisions must be made to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

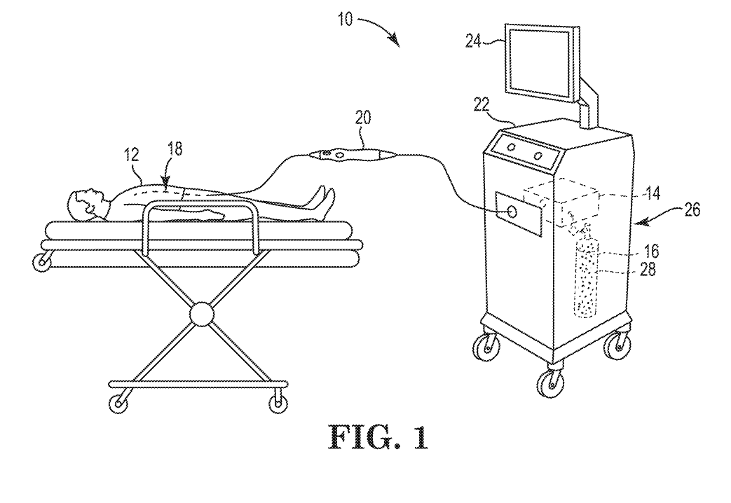

FIG. 1 is a schematic view of one embodiment of a cryogenic balloon catheter system 10 (also sometimes referred to as a “catheter system”) for use with a patient 12, which can be a human being or an animal. Although the catheter system 10 is specifically described herein with respect to the cryogenic balloon catheter system, it is understood and appreciated that other types of catheter systems and/or ablation systems can equally benefit by the teachings provided herein. For example, in certain non-exclusive alternative embodiments, the present disclosure can be equally applicable for use with any suitable types of ablation systems and/or any suitable types of catheter systems. Thus, the specific reference herein to use as part of the cryogenic balloon catheter system is not intended to be limiting in any manner.

The design of the catheter system 10 can be varied. In certain embodiments, such as the embodiment illustrated in FIG. 1, the catheter system 10 can include one or more of a control system 14, a fluid source 16 (e.g., one or more fluid containers), a balloon catheter 18, a handle assembly 20, a control console 22, a graphical display 24 (also sometimes referred to as a graphical user interface or “GUI”) and a residual fluid measurement system 26. It is understood that although FIG. 1 illustrates the structures of the catheter system 10 in a particular position, sequence and/or order, these structures can be located in any suitably different position, sequence and/or order than that illustrated in FIG. 1. It is also understood that the catheter system 10 can include fewer or additional components than those specifically illustrated and described herein.

In various embodiments, the control system 14 is configured to monitor and control the various processes of the ablation procedure. More specifically, the control system 14 can monitor and control release and/or retrieval of a cryogenic fluid 28 to and/or from the balloon catheter 18. The control system 14 can also control various structures that are responsible for maintaining and/or adjusting a flow rate and/or pressure of the cryogenic fluid 28 that is released to the balloon catheter 18 during a cryoablation procedure. In such embodiments, the catheter system 10 delivers ablative energy in the form of cryogenic fluid 28 to cardiac tissue of the patient 12 to create tissue necrosis, rendering the ablated tissue incapable of conducting electrical signals. Additionally, in various embodiments, the control system 14 can control activation and/or deactivation of one or more other processes of the balloon catheter 18. Further, or in the alternative, the control system 14 can receive data and/or other information (also sometimes referred to as “sensor output”) from various structures within the catheter system 10. In various embodiments, the control system 14 and the GUI 24 and/or the residual fluid measurement system 26 can be electrically connected and/or coupled. In some embodiments, the control system 14 can receive, monitor, assimilate and/or integrate any sensor output and/or any other data or information received from any structure within the catheter system 10 in order to control the operation of the balloon catheter 18. Still further, or in the alternative, the control system 14 can control positioning of portions of the balloon catheter 18 within the body of the patient 12, and/or can control any other suitable functions of the balloon catheter 18.

The fluid source 16 (also sometimes referred to as “fluid container 16”) can include one or more fluid container(s) 16. It is understood that while one fluid container 16 is illustrated in FIG. 1, any suitable number of fluid containers 16 may be used. The fluid container(s) 16 can be of any suitable size, shape and/or design. The fluid container(s) 16 contains the cryogenic fluid 28, which is delivered to the balloon catheter 18 with or without input from the control system 14 during a cryoablation procedure. Additionally, the type of cryogenic fluid 28 that is used during the cryoablation procedure can vary. In one non-exclusive embodiment, the cryogenic fluid 28 can include liquid nitrous oxide. In another non-exclusive embodiment, the cryogenic fluid 28 can include liquid nitrogen. However, any other suitable cryogenic fluid 28 can be used.

The design of the balloon catheter 18 can be varied to suit the specific design requirements of the catheter system 10. As shown, the balloon catheter 18 is inserted into the body of the patient 12 during the cryoablation procedure. In one embodiment, the balloon catheter 18 can be positioned within the body of the patient 12 using the control system 14. Stated in another manner, the control system 14 can control positioning of the balloon catheter 18 within the body of the patient 12. Alternatively, the balloon catheter 18 can be manually positioned within the body of the patient 12 by a qualified health professional (also referred to herein as an “operator” or “user”). As used herein, health care professional, operator or user can include a physician, a physician's assistant, a nurse and/or any other suitable person and/or individual. In certain embodiments, the balloon catheter 18 is positioned within the body of the patient 12 utilizing at least a portion of the sensor output that is received from the balloon catheter 18. For example, in various embodiments, the sensor output is received by the control system 14, which can then provide the operator with information regarding the positioning of the balloon catheter 18. Based at least partially on the sensor output feedback received by the control system 14, the operator or user can adjust the positioning of the balloon catheter 18 within the body of the patient 12 to ensure that the balloon catheter 18 is properly positioned relative to target cardiac tissue. While specific reference is made herein to the balloon catheter 18, as noted above, it is understood that any suitable type of medical device and/or catheter may be used.

The handle assembly 20 is handled and used by the operator or user to operate, position and control the balloon catheter 18. The design and specific features of the handle assembly 20 can vary to suit the design requirements of the catheter system 10. In the embodiment illustrated in FIG. 1, the handle assembly 20 is separate from, but in electrical and/or fluid communication with the control system 14, the fluid container 16, the graphical display 24 and/or the residual fluid measurement system 26. In some embodiments, the handle assembly 20 can integrate and/or include at least a portion of the control system 14 within an interior of the handle assembly 20. It is understood that the handle assembly 20 can include fewer or additional components than those specifically illustrated and described herein.

In the embodiment illustrated in FIG. 1, the control console 22 includes at least a portion of the control system 14, the fluid container 16, the GUI 24 and/or the residual fluid measurement system 26. However, in alternative embodiments, the control console 22 can contain additional structures not shown or described herein. Still alternatively, the control console 22 may not include various structures that are illustrated within the control console 22 in FIG. 1. For example, in certain non-exclusive alternative embodiments, the control console 22 does not include the GUI 24.

In various embodiments, the GUI 24 is electrically connected to the control system 14 and/or the residual fluid measurement system 26. Additionally, the GUI 24 provides the operator or user of the catheter system 10 with information that can be used before, during and after the cryoablation procedure. For example, the GUI 24 can provide the operator or user with information based on the sensor output, and any other relevant information that can be used before, during and after the cryoablation procedure. The specifics of the GUI 24 can vary depending upon the design requirements of the catheter system 10, or the specific needs, specifications and/or desires of the operator or user.

In one embodiment, the GUI 24 can provide static visual data and/or information to the operator or user. In addition, or in the alternative, the GUI 24 can provide dynamic visual data and/or information to the operator or user, such as video data or any other data that changes over time, e.g., during an ablation procedure. Further, in various embodiments, the GUI 24 can include one or more colors, different sizes, varying brightness, etc., that may act as alerts to the operator or user. Additionally, or in the alternative, the GUI 24 can provide audio data or information to the operator or user.

The residual fluid measurement system 26 measures a quantity of the cryogenic fluid 28 remaining within the fluid container(s) 16 during one or more cryoablation procedures (also sometimes referred to as “residual fluid quantity”). In particular, the residual fluid measurement system 26 is specifically configured to provide the operator or user of the catheter system 10 with information to determine residual cryoablation information. As used herein, residual cryoablation information can include a number of cryoablations that may be performed and/or an amount of time to perform cryoablations based on the residual fluid quantity. In other words, the residual fluid measurement system 26 can determine whether there is a sufficient residual fluid quantity within the fluid container 16 to perform a certain number of cryoablation procedures and/or a certain amount of time to perform cryoablation procedures.

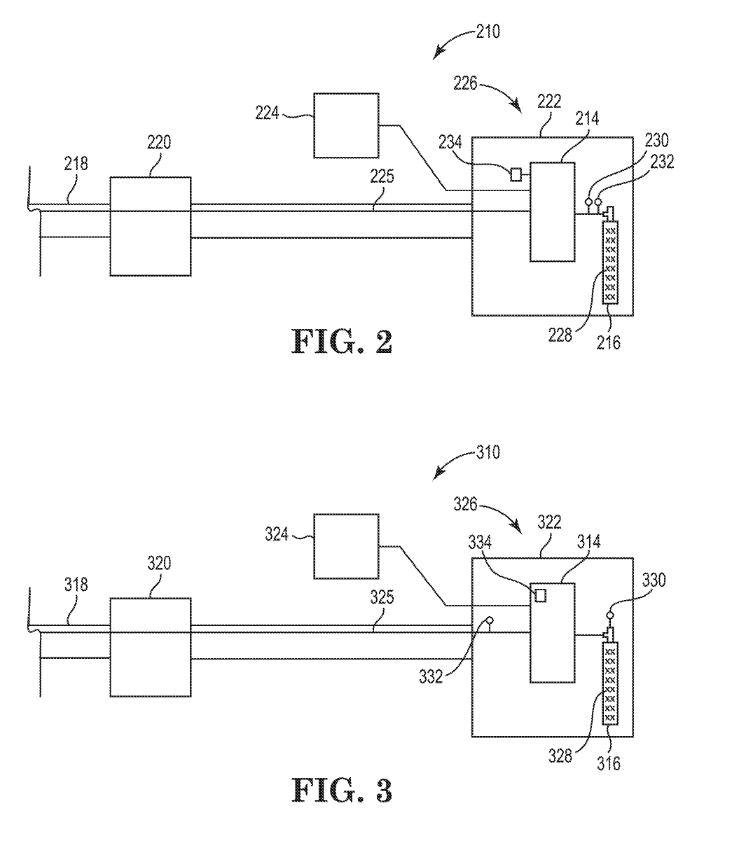

In the embodiment illustrated in FIG. 1, at least a portion of the residual fluid measurement system 26 is positioned at a location within the control console 22. The residual fluid measurement system 26 can be positioned at any suitable location within the control console 22. Alternatively, the residual fluid measurement system 26 can be positioned at any suitable location outside of the control console 22. Further, portions of the residual fluid measurement system 26 can be positioned partially within and/or outside the control console 22. Additionally, and/or alternatively, the residual fluid measurement system 26 can be positioned at any other suitable location within the catheter system 10. The specific components and operations of the residual fluid measurement system 26 will be described in greater detail herein below in relation to the embodiment illustrated in FIG. 2.

FIG. 2 is a simplified schematic side view of an embodiment of a portion of the catheter system 210 including another embodiment of the residual fluid measurement system 226. In the embodiment illustrated in FIG. 2, the catheter system 210 includes the control system 214, the fluid container 216, the balloon catheter 218, the handle assembly 220, the control console 222, the GUI 224, a fluid injection line 225 and the residual fluid measurement system 226.

The fluid injection line 225 functions as a conduit through which the cryogenic fluid 228 is delivered from the fluid container 216 to the balloon catheter 218 during the cryoablation procedure. The design of fluid injection line 225 can vary. In the embodiment illustrated in FIG. 2, a portion of the fluid injection line 225 is shown to extend from the fluid container 216 to the balloon catheter 218. In alternative embodiments, the fluid injection line 225 can be connected to and/or extend through other structures and/or components of the catheter system 210.

The residual fluid measurement system 226 enables the operator or user to more accurately assess whether there is sufficient residual fluid quantity remaining within the fluid container 216 to successfully complete cryoablation procedures. The design of the residual fluid measurement system 226 can be varied. As shown in this embodiment illustrated in FIG. 2, the residual fluid measurement system 226 can include one or more of a first fluid sensor 230, a second fluid sensor 232 and a controller 234. Alternatively, the residual fluid measurement system 226 can include additional components or fewer components than those specifically illustrated and described herein.

In certain embodiments, such as the embodiment illustrated in FIG. 2, the fluid container 216 can include the residual fluid quantity of the cryogenic fluid 228 remaining within the fluid container 216. The residual fluid quantity of the cryogenic fluid 228 remaining with the fluid container 216 can vary at any moment depending on the number of cryoablation procedures performed and/or the amount of time to perform the cryoablation procedures.

The first fluid sensor 230 and/or the second fluid sensor 232 can monitor various properties of the cryogenic fluid 228 that is being used during the cryoablation procedure. The design of the first fluid sensor 230 and/or the second fluid sensor 232 can vary. It is recognized that the terms “first fluid sensor 230” and “second fluid sensor 232” can be used interchangeably. In other words, either sensor 230, 232, can be the first fluid sensor 230 or the second fluid sensor 232. Additionally, the first fluid sensor 230 and/or the second fluid sensor 232 can monitor various properties of the cryogenic fluid 228 within the fluid container 216. The first fluid sensor 230 and/or the second fluid sensor 232 can include one or more of a pressure sensor, a weight sensor and/or a flow rate sensor, as non-exclusive examples.

It is understood that although the first fluid sensor 230 and/or the second fluid sensor 232 illustrated in FIG. 2 are shown in certain locations within the catheter system 210, the first fluid sensor 230 and/or the second fluid sensor 232 can be positioned in any suitable location within the catheter system 210, including other than those illustrated in FIG. 2. In certain embodiments, the first fluid sensor 230 and/or the second fluid sensor 232 can be positioned adjacent to the fluid container 216 in order to monitor the fluid pressure immediately after the cryogenic fluid 228 exits and/or is released from the fluid container 216. In some embodiments, the first fluid sensor 230 and/or the second fluid sensor 232 can monitor the fluid pressure of the cryogenic fluid 228 within the fluid container 216. For example, the first fluid sensor 230 and/or the second fluid sensor 232 can include a pressure gauge that monitors the fluid pressure of the cryogenic fluid 228 within the fluid container 216. In other embodiments, the first fluid sensor 230 and/or the second fluid sensor 232 can be positioned adjacent to the fluid container 216 in order to monitor the weight of the cryogenic fluid 228 within the fluid container 216. For example, the first fluid sensor 230 and/or the second fluid sensor 232 can include a scale that monitors the weight of the fluid container 216, the cryogenic fluid 228 within the fluid container 216, or both. In yet other embodiments, the first fluid sensor 230 and/or the second fluid sensor 232 can be positioned adjacent to the fluid container to monitor a fluid flow rate of the cryogenic fluid 228 exiting and/or released from the fluid container 216 during the cryoablation procedure.

The first fluid sensor 230 and/or second fluid sensor 232 can transmit or send electronic and/or other signals, e.g., sensor output, to the controller 234. In other words, the first fluid sensor 230 can generate first sensor output and the second fluid sensor 232 can generate second sensor output. For ease of reference, the sensor output generated by the first fluid sensor 230 and/or the second fluid sensor 232, i.e., first sensor output, second sensor output, etc., is generally referred to herein as “sensor output.” As one non-exclusive example, sensor output can include the residual fluid quantity. In this example, the first fluid sensor 230 and/or the second fluid sensor 232 can sense and/or measure the residual fluid quantity based on a pressure of the cryogenic fluid 228 within the fluid container 216, a weight of the cryogenic fluid 228 therein, and/or a combination of the pressure and/or weight. As another non-exclusive example, the sensor output can also include consumption information.

As used herein, consumption information can include an actual fluid flow rate of the cryogenic fluid 228 or an average fluid flow rate of the cryogenic fluid 228 during the cryoablation procedures. The actual fluid flow rate can include the flow rate of the cryogenic fluid 228 at any specific time during the cryoablation procedure. The average fluid flow rate includes the average flow rate of the cryogenic fluid 228 measured over a predetermined time period and/or over the entire cryoablation procedure. Alternatively, consumption information may include any other suitable set value and/or measurement related to the cryogenic fluid 228 and/or its consumption. In various embodiments, the first fluid sensor 230 and/or the second fluid sensor 232 can sense and/or measure residual fluid quantity and/or consumption information, and transmit or send sensor output to the controller 234.

Additionally, the first fluid sensor 230 and/or the second fluid sensor 232 can sense and/or measure residual fluid quantity and/or consumption information via any suitable manner and/or method. In the embodiment illustrated in FIG. 2, the first fluid sensor 230 and/or the second fluid sensor 232 can be positioned on and/or within the fluid injection line 225 adjacent to the fluid container 216. In this embodiment, the first fluid sensor 230 and/or the second fluid sensor 232 can directly sense and/or measure residual fluid quantity and/or consumption information. As used herein, the term “directly” sense means that the first fluid sensor 230 and/or the second fluid sensor 232 is in direct contact with the cryogenic fluid 228 during the cryoablation procedure.

In other embodiments, the first fluid sensor 230 and/or the second fluid sensor 232 may be positioned on and/or within the fluid container 216.

In alternative embodiments, the first fluid sensor 230 and/or the second fluid sensor 232 may not be positioned on and/or within the fluid injection line 225 and/or the fluid container 216. In such alternative embodiments, the first fluid sensor 230 and/or the second fluid sensor 232 can indirectly sense and/or measure residual fluid quantity and/or consumption information. As used herein, the term “indirectly” sense means that the first fluid sensor 230 and/or the second fluid sensor 232 is not in direct contact with the cryogenic fluid 228 during the cryoablation procedure.

As one non-exclusive example, the first fluid sensor 230 and/or the second fluid sensor 232 may be located outside of the fluid injection line 225 and/or the fluid container 216, such as in the control console 222, when using a scale, for example. In such embodiments, the first fluid sensor 230 and/or the second fluid sensor 232 can sense and/or measure residual fluid quantity and/or consumption information due to a change in pressure and/or weight of the fluid container 216 or the cryogenic fluid 228 within the fluid container 216, or both. Alternatively, the first fluid sensor 230 and/or the second fluid sensor 232 can indirectly sense and/or measure the residual fluid quantity and/or consumption information via any suitable manner or method.

The controller 234 is configured to receive and/or process the sensor output transmitted or sent from the first fluid sensor 230 and/or the second fluid sensor 232. In various embodiments, the first fluid sensor 230 and/or the second fluid sensor 232 can be electrically connected and/or coupled to the controller 234. The design of the controller 234 can vary. In some embodiments, the sensor output directly sensed and transmitted by the first fluid sensor 230 and/or the second fluid sensor 232 can then be processed by the controller 234 to determine residual cryoablation information. In other embodiments, the sensor output indirectly sensed and transmitted by the first fluid sensor 230 and/or the second fluid sensor 232 can also then be processed by the controller 234. In such other embodiments, the controller 234 can process the sensor output indirectly sensed to determine residual cryoablation information. The controller 234 can process the sensor output, whether directly or indirectly sensed, via any suitable method, including algorithm, for example.

In some embodiments, the controller 234 can process a pressure of the cryogenic fluid 228 within the fluid container 216, a weight of the cryogenic fluid 228 therein, and/or a combination of the pressure and/or weight sent and/or transmitted by the first fluid sensor 230 and/or second fluid sensor 232 to determine the residual fluid quantity. In other embodiments, the controller 234 can process the flow rate of the cryogenic fluid 228 over a predetermined time period and/or over the entire cryoablation procedure sent and/or transmitted by the first fluid sensor 230 and/or the second fluid sensor 232 to determine the average fluid flow rate.

Further, although shown in the embodiment illustrated in FIG. 2 as a separate structure, the controller 234 can be included as part of the control system. In other embodiments, the controller 234 can be separate from the control system 214.

FIG. 3 is a simplified schematic side view of another embodiment of a portion of the catheter system 310 including still another embodiment of the residual fluid measurement system 326. In the embodiment illustrated in FIG. 3, the catheter system 310 includes the control system 314, the fluid container 316, the balloon catheter 318, the handle assembly 320, the control console 322, the GUI 324, the fluid injection line 325 and residual fluid measurement system 326. However, in this embodiment, the residual fluid measurement system 326 also includes the GUI 324. The GUI 324 of the residual fluid measurement system 326 can provide the residual cryoablation information determined by the controller 334 to the operator or user. As non-exclusive examples, the GUI 324 can provide the residual cryoablation information to the operator or user visually by picture, data, numbers or percentages. In other non-exclusive embodiments, the GUI 324 can provide the operator or user with one or more of the following: a number of cryoablation procedures that may be performed based on the residual fluid quantity and/or an amount of time to perform cryoablation procedures based on the residual fluid quantity. However, any other suitable manner can be used by the GUI 324 to effectively provide and/or notify the operator or user of the residual cryoablation information.

Additionally, in the embodiment illustrated in FIG. 3, the residual fluid measurement system 326 also includes the first fluid sensor 330 and the second fluid sensor 332, however, the first fluid sensor 330 includes a pressure gauge 330. In this embodiment, the first fluid sensor 330 is located and/or positioned on the fluid container 316 to allow the pressure gauge 330 to monitor the fluid pressure of the cryogenic fluid 328 within the fluid container 316. In alternative embodiments, the first fluid sensor 330 can be located and/or positioned on and/or within any suitable structure and/or component of the catheter system 310. Further, the second fluid sensor 332 is located and/or positioned on the fluid injection line 325, but away from, i.e., not adjacent, to the fluid container 316. Alternatively, the second fluid sensor 332 can be located and/or positioned on and/or within any suitable structure and/or component of the catheter system 310. Moreover, in this embodiment, the controller 334 is integrated and/or included as part of the control system 314.

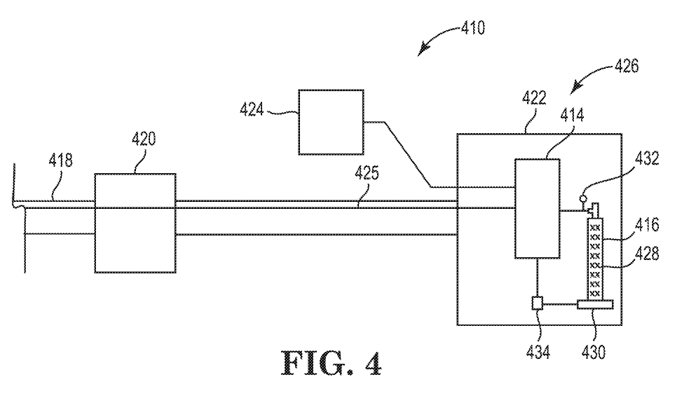

FIG. 4 a simplified schematic side view of still another embodiment of a portion of the catheter system 410 including yet another embodiment of the residual fluid measurement system 426. In the embodiment illustrated in FIG. 4, the catheter system 410 includes the control system 414, the fluid container 416, the balloon catheter 418, the handle assembly 420, the control console 422, the GUI 424, the fluid injection line 425 and the residual fluid measurement system 426. However, in the embodiment illustrated in FIG. 4, the residual fluid measurement system 426 includes the first fluid sensor 430 and the second fluid sensor 432, wherein the first fluid sensor 430 includes a scale 430. The scale 430 can be electrically connected to the controller 434. In this embodiment, the fluid container 416 can be positioned on and/or adjacent to the scale 430 to allow the scale 430 to monitor the weight of the fluid container 416, the cryogenic fluid 428 within the fluid container 416, or both. Further, the first fluid sensor 430 is located and/or positioned within the control console 422. Alternatively, the first fluid sensor 430 can be positioned and/or located at any suitable location within the control console 422.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

I claim:

1. A cryoablation system comprising:
a balloon catheter including a cryoballoon for delivering ablative energy to patient tissue;
a fluid container operatively coupled to the balloon catheter and configured to supply cryogenic fluid to the cryoballoon and to store a residual fluid quantity;
a first fluid sensor configured to sense the residual fluid quantity, the first fluid sensor generating a first sensor output;
a second fluid sensor configured to sense fluid consumption information representing an amount of fluid consumption by the balloon catheter during one or more ablation procedures, the second fluid sensor generating a second sensor output; and
a controller configured to receive the first sensor output and the second sensor output and to determine residual cryoablation information based at least in part on the first sensor output and the second sensor output.

2. The cryoablation system of claim 1, wherein the residual cryoablation information includes a number of cryoablation procedures that may be performed with the residual fluid quantity.

3. The cryoablation system of claim 1, wherein the residual cryoablation information includes an amount of time available to perform one or more cryoablation procedures using the residual fluid quantity.

4. The cryoablation system of claim 1, wherein the first fluid sensor includes a pressure sensor or a weight sensor.

5. The cryoablation system of claim 1, wherein the second fluid sensor includes a flow rate sensor.

6. The cryoablation system of claim 5, wherein the fluid consumption information includes an actual fluid flow rate.

7. The cryoablation system of claim 5, wherein the fluid consumption information includes an average fluid flow rate.

8. The cryoablation system of claim 1, further comprising a graphical user interface including a display configured to display the residual cryoablation information.

9. A method for determining residual cryoablation information for a fluid container having a residual fluid quantity, the method comprising:
generating a first sensor output with a first fluid sensor; sending the first sensor output to a controller; and
determining the residual cryoablation information with the controller based at least in part upon the first sensor output.

10. The method of claim 9, wherein the residual cryoablation information includes a number of cryoablation procedures that may be performed with the residual fluid quantity.

11. The method of claim 9, wherein the residual cryoablation information includes an amount of time to perform cryoablation procedures with the residual fluid quantity.

12. The method of claim 9, further comprising generating a second sensor output with a second fluid sensor.

13. The method of claim 12, further comprising sending the second sensor output generated by the second connection sensor to the controller.

14. The method of claim 13, further comprising determining the residual cryoablation information based at least partially upon the second sensor output with the controller.

15. The method of claim 14, further comprising displaying the residual cryoablation information on a display of a graphical user interface.

16. A residual fluid measurement system for determining residual cryoablation information for a fluid container having a residual fluid quantity, the residual fluid measurement system comprising:
a first fluid sensor configured to sense the residual fluid quantity, the first fluid sensor generating a first sensor output; and
a controller configured to receive the first sensor output and determine the residual cryoablation information based at least in part on the first sensor output.

17. The residual fluid measurement system of claim 16, wherein the residual cryoablation information includes a number of cryoablation procedures that may be performed.

18. The residual fluid measurement system of claim 16, wherein the residual cryoablation information includes an amount of time available to perform one or more cryoablation procedures.

19. The residual fluid measurement system of claim 16, further comprising a second fluid sensor that senses fluid consumption information, the second fluid sensor generating a second sensor output.

20. The residual fluid measurement system of claim 19, wherein the controller receives the second sensor output, the controller determining the residual cryoablation information based at least in part on the second sensor output.
